(19) United States
(12) Patent Application Publication
(10) Pub. No.: US 2023/0091365 A1
(43) Pub. Date: Mar. 23, 2023

(54) Automated Self-Actuated Medicine Injection System

(71) Applicant: Innovative Health Strategies LLC, Peoria, IL (US)

(72) Inventors: Charles Vaughn Bandoian, Peoria, IL (US); Mohammad Shariq Imtiaz, Peoria, IL (US); Thomas Joseph Santoro, MD, Peoria, IL (US)

(73) Assignee: Innovative Health Strategies LLC, Peoria, IL (US)

(21) Appl. No.: 17/952,200

(22) Filed: Sep. 23, 2022

Related U.S. Application Data
(60) Provisional application No. 63/247,590, filed on Sep. 23, 2021.

(51) Int. Cl. A61M 5/145; A61M 5/20; A61M 5/142; A61M 5/158; A61M 5/168

(52) U.S. Cl. CPC A61M 5/14526; A61M 5/2046; A61M 5/14248; A61M 5/158; A61M 5/16881

(57) ABSTRACT

A compact and smart wearable device is configured to automatically inject medicines into the wearer. Because hypodermic needles are only deployed when triggered, infection risks are eliminated. Necessary medicines are automatically deployed based on physiological or other data without human intervention to save lives. The wearable device can also be configured to inject more than one medicine into the wearer. And can be secured to the wearer with a band having inflatable balloons that can tighten and reposition the wearable device on the wearer

### CROSS-REFERENCE TO RELATED APPLICATIONS

This application claims the priority benefit of U.S. Provisional Patent Application No. 63/247,590 filed on Sep. 23, 2021, the disclosure of which is expressly incorporated herein in its entirety by reference.

### STATEMENT REGARDING FEDERALLY SPONSORED RESEARCH

Not Applicable

### PARTIES TO A JOINT RESEARCH AGREEMENT

Not Applicable

### REFERENCE TO APPENDIX

Not Applicable

### FIELD OF THE INVENTION

The field of the present invention generally relates to drug delivery systems and, more particularly, to automated drug delivery systems.

### BACKGROUND OF THE INVENTION

Catastrophic emergencies are acute medical conditions that require immediate treatment. Such acute medical conditions include, but are not limited to, drug overdose, diabetic coma precipitated by the relative lack of insulin, and anaphylactic shock in response to the bite of an insect to which one harbors a sever allergy. Effective treatment of these acute medical conditions frequently requires the immediate administration of medication needed to mitigate the cardiac, respiratory and/or metabolic consequences of the emergency. Because catastrophic emergencies may occur unwitnessed, evolve rapidly, and/or compromise cognitive as well as physical capacity, the most reliable treatment device should be automatic (i.e. involve no decision making or physical action on the part of the victim), specific (i.e. as selective as possible for the emergent condition), and predictive (i.e. should be triggered by physiological data that reliably predicts an impending catastrophic event).

Death by overdosing with opioids has increased by an alarming rate over the past decade and continues to rise. This increase in opioid related mortality is, in part, related to a lack of timely, efficient and effective treatment of the overdose.

Currently available automatic drug injection systems typically require embedded cannulas. These cannulas can become unintentionally dislodges or can cause infection. Additionally, currently available automatic drug injection systems also typically require human intervention. Such as, for example, pressing an activation button. Furthermore, currently available drug delivery systems are bulky and unwearable. Even when they are wearable, they are obtrusive and inconvenient to wear.

Accordingly, there is a need for a self-activating drug delivery system that can store and deliver specific medicines for specific conditions on-demand and without human intervention.

### SUMMARY OF THE INVENTION

Disclosed are devices and methods which overcome at least some of the above-identified problems of the prior art. Disclosed is a wearable device for automatically injecting a fluid into a wearer of the wearable device. The wearable device comprising, in combination, an enclosure, an attachment for securing the enclosure to the wearer, at least one injection cylinder within the enclosure, and an injection piston within the injection cylinder configured for linear movement within the injection cylinder. The injection piston includes opposed open and closed ends and forms an internal fluid compartment containing the fluid to be injected. A plunger is located within the fluid compartment between the open end of the injection piston and the fluid to be injected and selectively movable within the fluid compartment toward the closed end of the injection piston. At least one hypodermic needle is carried by the injection piston for insertion into the wearer and in fluid flow communication with the fluid to be injected within the internal fluid compartment. A force acting on the plunger initially moves the injection piston to insert the at least one hypodermic needle into the wearer and then moves the plunger within the fluid compartment to inject at least a portion of the fluid through the at least one hypodermic needle.

Also disclosed is a wearable device for automatically injecting a fluid into a wearer of the wearable device, the wearable device comprising, in combination, an enclosure holding a plurality of vials each holding a different fluid to be injected into the wearer, an attachment for securing the enclosure to the wearer, a plurality of vial plungers each within a separate one of the plurality of vials and configured to selectively move the fluid out of the opposite end of the separate one of the plurality of vials when a force is applied to the vial plunger, a plurality of cartridges of compressed gas, and a plurality of electric valves each associated with a separate one of the plurality of cartridges and configured for selectively allowing flow of the compressed gas from the associated cartridge to the associated plunger to supply a force on the associated plunger, and a plurality of needle cylinders each configured to receive the fluid from a separate one of the vials. Each of the plurality of needle cylinders includes a plunger movable within the associated needle cylinder and carrying at least one hypodermic needle for insertion into the wearer. The wearable device further comprises a plurality of fluid conduits each connecting an outlet of a separate one of the vials to the associated at least one hypodermic needle, and a plurality of compressed-gas conduits each connecting one of the plurality of electric valves with an separate one of the plurality of needle cylinders to selectively move the associated needle piston to insert the at least one hypodermic needle into the wearer when compressed gas is supplied to the associate one of the plurality of vials.

Further disclosed is a system for automatically injecting a fluid into a wearer of the system, the system comprising, in combination, a wearable device for automatically injecting a fluid into the wearer and including a container for holding the fluid and at least one hypodermic needle for insertion into the wearer, a band for securing the wearable device to the wearer and including at least one inflatable balloon, at least one cartridge of compressed gas, and at least one electric valve configured for selectively allowing flow of the compressed gas from the at least one cartridge to the at least one inflatable balloon to inflate the inflatable balloon.

From the foregoing disclosures and the following more detailed description of various preferred embodiments it will be apparent to those skilled in the art that the present invention provides a significant advance in the technology and art of automatic drug delivery systems. Particularly significant in this regard is the potential the invention affords for wearable automatic drug injection systems that require no human intervention and are unobtrusive and convenient to wear. Additionally, the systems utilize an objective measurement to determine when to activate drug delivery. For example in the case of opioid overdoses, respiratory impairment (i.e. low levels of oxygenated hemoglobin in the blood of the victim) is assessed by a reflectance oximeter or an oxygen-sensor to take the “guess work” out of when to administer the antidote. This also obviates the need for others to be present in order to treat an opioid-induced overdose and reduces false reporting. Additional features and advantages of various preferred embodiments will be better understood in view of the detailed description provided below.

### BRIEF DESCRIPTION OF THE DRAWINGS

These and further features of the present invention will be apparent with reference to the following description and drawing, wherein:

FIG. 1 is a schematic view of a wearable device for automatically injecting medicines into a wearer according to a first embodiment of the present invention.

FIG. 2 is a schematic view of a metering system of the wearable device of FIG. 1.

FIG. 3 is a schematic view of an alternative metering system of the wearable device of FIG. 1.

FIG. 4 is a top schematic view of a wearable device for automatically injecting medicines into a wearer according to a second embodiment of the present invention.

FIG. 5 is an enlarged side cross-sectional schematic view of a needle cylinder of the wearable device of FIG. 4.

FIG. 6 is schematic view of “watch” or wrist band for securing the wearable devices of FIGS. 1 to 4.

FIG. 7 is a schematic view of an inflation system for the of FIG. 6.

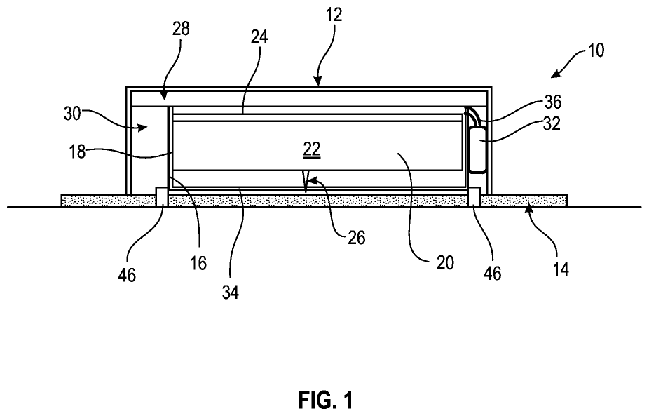

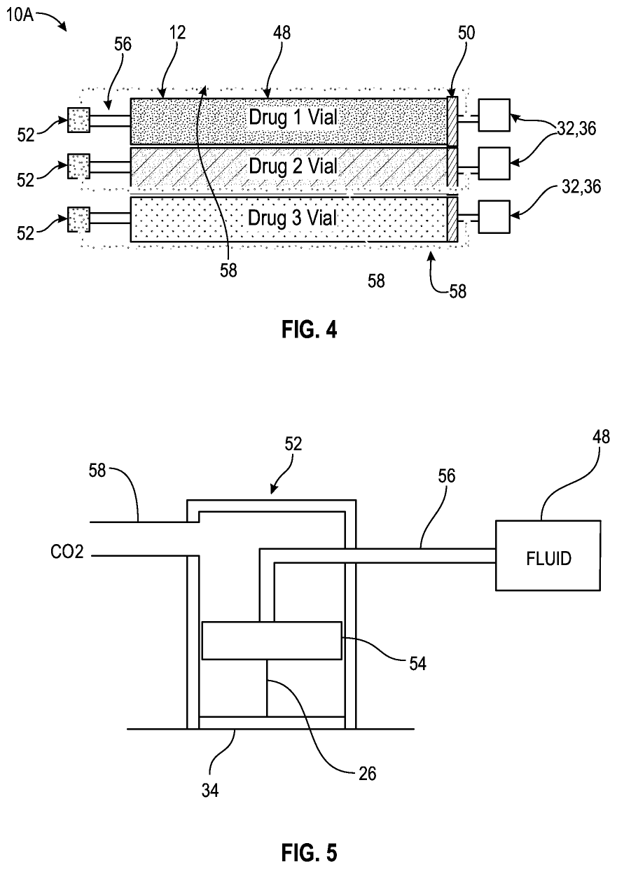

It should be understood that the appended drawings are not necessarily to scale, presenting a somewhat simplified representation of various preferred features illustrative of the basic principles of the invention. The specific design features of the wearable devices as disclosed herein, including, for example, specific dimensions and shapes of the various components will be determined in part by the particular intended application and use environment. Certain features of the illustrated embodiments have been enlarged or distorted relative to others to facilitate visualization and clear understanding. In particular, thin features may be thickened, for example, for clarity or illustration. All references to direction and position, unless otherwise indicated, refer to the orientation of the storage compartment support systems illustrated in the drawings. In general, up or upward refers to an upward direction generally within the plane of the paper in FIG. 4 and down or downward refers to a downward direction generally within the plane of the paper in FIG. 4. Also in general, forward or front refers to a direction extending out the plane of the paper in FIG. 1 and back or rear refers to a direction extending into the plane of the paper in FIG. 1.

### DETAILED DESCRIPTION OF CERTAIN PREFERRED EMBODIMENTS

It will be apparent to those skilled in the art, that is, to those who have knowledge or experience in this area of technology, that many uses and design variations are possible for the wearable devices for injecting medicines and other liquids into wearers disclosed herein. The following detailed discussion of various alternative and preferred embodiments will illustrate the general principles of the invention with an embodiment configured to automatically injecting an antidote such as, for example NARCAN or the like to prevent death by overdosing with opioid. Other embodiments suitable for other applications will be apparent to those skilled in the art given the benefit of this disclosure such as for example, but not limited to, automatically injecting insulin to prevent death by a diabetic coma precipitated by the relative lack of insulin, automatically injecting adrenaline to prevent death by anaphylactic shock due to a bite by an insect to which one harbors a sever allergy, injecting both a pain killer such as fentanyl and an overdose antidote such as NARCAN to reduce pain of a severe injury such as on a battlefield, and to inject one or more antidotes to exposure of chemical or biological weapons on a battlefield.

FIG. 1 illustrates an exemplary wearable device (10) for automatically injecting a fluid into a wearer of the wearable device (10). The fluid is typically a liquid but and is typically a medicine or drug but can alternatively be any other suitable fluid. The illustrated wearable device (10) includes a case or enclosure (12), an attachment (14) for securing the enclosure (12) to the wearer, at least one injection cylinder (16) within the enclosure (12), an injection piston (18) within the injection cylinder (16) configured for linear movement within the injection cylinder (16), wherein the injection piston (18) includes open and closed ends and forms an internal fluid compartment (20) containing the fluid (22) to be injected, a plunger (24) within the fluid compartment (20) between the open end of the injection piston (18) and the fluid (22) to be injected and selectively movable within the fluid compartment (20) toward the closed end of the injection piston (18), at least one hypodermic needle (26) carried by the injection piston (18) for insertion into the wearer and in fluid flow communication with the fluid (22) to be injected within the fluid compartment (20). When a force is provided on the plunger (24), the plunger (24) initially moves the injection piston (18) to insert the at least one hypodermic needle (26) into the wearer, and then moves the plunger (24) within the fluid compartment (20) to inject at least a portion of the fluid (22) through the at least one hypodermic needle (26). It is noted that the illustrated wearable device (10) can alternatively have any other suitable configuration as described in more detail hereinbelow.

The illustrated enclosure (12) is generally a rectangular-shaped box sized and shaped to enclose components of the wearable device (10) as described in more detail herein below. The enclosure (12) is preferably sized as small as possible while allowing the wearable device (10) to perform as described herein. A top portion of the enclosure provides an electronics compartment for containing electronics for operating the wearable device (10) such as a microcontroller (28). A first side portion of the enclosure (12) provides a battery compartment for containing one or more batteries I30) for powering the electrical components of wearable device (10). A second side portion of the enclosure (12) provides a compressed-gas compartment for containing one or more cartridges (32) of compressed gas for driving the fluid (22). A central portion of the container provides the injection cylinder (16) in which the injection piston (18) linearly moves. The injection cylinder (16) extends vertically and has a circular cross-sectional shape but aby other suitable configuration and/or shape can alternatively be utilized. The bottom of the injection cylinder (16) is closed with a protective membrane (34) that seals closed the injection cylinder (16) to prevent contamination of the hypodermic needle (26) prior to use but is pierceably by the hypodermic needle(s) (26) upon downward movement of the hypodermic needle(s) (26). It is noted that the enclosure (12) can be formed of any suitable material or materials. The enclosure (12) can also be formed by an number of separate together in any suitable manner such as for example, but not limited to, mechanical fasteners. It is also noted that the enclosure (12) can alternatively have any other suitable size, shape, and/or configuration.

The illustrated attachment (14) for securing the enclosure (12) to the wearer is a layer of adhesive provided at the bottom surface of the enclosure (12) and the protective membrane (34). The illustrated layer of adhesive can be pierced by the hypodermic needle(s) (26). The layer of adhesive can be pressed against the wearer's skin at a desired injection site or location in order to secure the enclosure (12) thereto. The layer of adhesive can be of any suitable type. It is noted that the attachment (14) can alternatively be of any suitable type such as, for example, but not limited to, a strap, belt, sleeve, portion of clothing, clothing, or the like.

The illustrated at least one injection cylinder (16) is centrally located within the enclosure (12) and has a closed top end and an open bottom end closed by the membrane (34). The illustrated embodiment has a single fluid cylinder that is vertically oriented. The illustrated injection cylinder (16) is circular in cross-section but can have any other suitable shape to cooperate with the injection or fluid piston (18) therein. It is noted that the Injection cylinder (16) can alternatively have any other suitable size, shape, and/or configuration.

The illustrated injection or fluid piston (18) is located within the fluid cylinder (16) and is configured for longitudinal liner movement in the vertical direction within the injection cylinder (16). The illustrated fluid piston (18) is cylindrically-shaped having a hollow interior cavity or fluid compartment (20) for holding a desired amount of the fluid (22) to be injected. The illustrated fluid compartment (20) is circular in cross-section but can have any other suitable shape to cooperate with the injection plunger (24) therein. The illustrated fluid compartment (20) is closed at the bottom end except for the hypodermic needle(s) (26) and open at the top end. The illustrated injection piston i(18) s sized to closely match the inner wall of the injection cylinder (16) to form a fluid tight seal therebetween while allowing movement of the injection piston (18) within the injection cylinder (16). The injection piston (18) can be provided with a seal at its outer edge if desired. The injection piston (18) divides the injection cylinder (16) into a first or upper portion which selectively receives compressed gas and a second or lower portion or lower compartment that houses the hypodermic needle(s) (26). It is noted that the injection piston (18) can alternatively have any other suitable size, shape, and/or configuration.

The illustrated plunger (24) is located within the fluid compartment (20) between the open end of the injection piston (18) and the fluid to be injected and selectively movable within the fluid compartment (20) toward the closed end of the injection piston (18). The illustrated plunger (24) is sized to closely match the side wall of the fluid compartment(20) to form a fluid tight seal therebetween while allowing linear movement of the plunger (24) within the injection cylinder (16). The plunger (24) can be provided with a seal at its outer edge if desired. The illustrated plunger (24) divides the fluid compartment (20) into a first or upper portion which selectively receives compressed gas and a second or lower portion that holds the fluid (22) to be injected. It is noted that the plunger (24) can alternatively have any other suitable size, shape, and/or configuration.

The illustrated at least one hypodermic needle (26) is secured to and carried by the injection piston (18) for insertion into the wearer. The illustrated embodiment includes a single hypodermic needle (26) that downwardly extends from the injection piston (18) into the needle compartment of the injection cylinder (16). The illustrated hypodermic needle (26) extends through the bottom wall of injection piston (18) and has an interior passage that extends entirely through so that the fluid to be injected can pass entirely through the hypodermic needle (26) from the upper end within fluid compartment to and out the lower tip of the hypodermic needle (26) injected into the wearer. It is noted that the at least one hypodermic needle (26) can alternatively have any other suitable size, shape, and/or configuration.

The illustrated embodiment also includes a pressurization system operably connected to the upper portion of the injection cylinder (16) above the plunger (24) for selectively driving the injection piston (18) and/or plunger (24) in a downward direction. The illustrated pressurization system includes the at least one compressed gas cartridge (32) located within compressed gas storage compartment within the enclosure (12) and at least one electric control valve (36) connecting the compressed gas cartridge(s) (32) with the upper portion of the injection cylinder (16). The compressed gas can be compressed air or compressed CO2 but any other suitable compressed gas can alternatively be utilized. The control valve(s) (36) is operatively connected to the microcontroller (28) to selectively open and close the control valve(s) (36) to control the flow of compressed gas from the compressed gas cartridge(s) (32). It is noted that the pressurization system can alternatively have any other suitable size, shape, and/or configuration. For example, but not limited to, the pressurized gas can be replaced with other energy sources, e.g. combustible chemical, mechanical or other electric sources etc. With the cartridge style gas container (or other energy source) and the wearable device (10) can be configured for rapid redeployment.

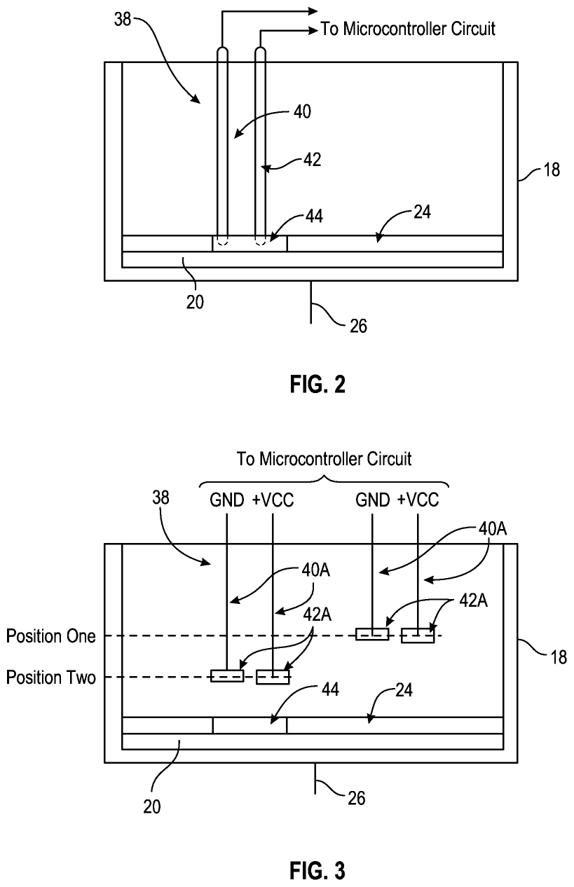

As best shown in FIG. 2, the plunger (24) is preferably provided with a metering system including a plunger position sensor (38) for controlling the quantity of fluid injected. The illustrated plunger position sensor (38) provides a continuous analog signal of the position of the plunger (24) to the microcontroller. The microcontroller (28) can determine the volume of fluid injected using an algorithm and the plunger position signal. The illustrated plunger position sensor (38) includes vertically extending wires (40) embedded in the wall of the injection piston (18), a vertically extending exposed contact (42) on an inner surface of the fluid compartment, (20) and an exposed conducting surface (44) on an outer edge of the plunger (24) sized and shaped to be in contact with the embedded wires (40) and the exposed contact as the plunger 924) moves within the fluid compartment (20). Upper ends of the embedded wires (40) and the exposed contact (42) are in electrical connection with the circuit of the microcontroller (28). It is noted that the plunger position sensor (38) can alternatively have any other suitable configuration.

FIG. 3 discloses an alternative plunger position sensor (38A). The illustrated alternative plunger position sensor (38A) provides one or more discrete signals of the position of the plunger (24) to the microcontroller (28). The illustrated embodiment includes two discrete positions but one or more than two positions can be utilized. The microcontroller (28) can determine the volume of fluid injected using an algorithm and the plunger position signal (38A). The illustrated alternative plunger position sensor (38A) includes vertically extending wires (40A) embedded in the wall of the injection piston (18), a pair of laterally spaced-apart exposed contacts (42A) on an inner surface of the fluid compartment (20) at each position to be sensed, and a conducting surface (44A) on an outer edge of the plunger (24) sized and shaped to be in contact with the pair of exposed contacts (42A) as the plunger (24) is at one of the discrete positions of the pair exposed contacts(42A). Lower ends of the embedded wires (40A) are each connected to one of the exposed contacts (42A). Upper ends of the embedded wires (40A) are in electrical connection with the circuit of the microcontroller (28). It is noted that the alternative plunger position sensor (38A) can alternatively have any other suitable configuration.

The control system for the illustrated wearable device (10) includes the micro controller (28) which is in communication with the battery (30) for powering components requiring electrical power, the control valve (36) of the pressurization system for opening and closing the control valve (36), the piston position sensor (38) for determining the amount of fluid injected into the wearer, and at least on sensor or electrode (46) for receiving biometric data regarding the wearer. The at least one sensor or electrode (46) is embedded in the bottom of the of the enclosure (12). Wires from the at least one sensor extend upward to the electronics compartment to the circuit of the microcontroller (28) located therein. The at least one sensor (46) provides biometric data to the microcontroller (28) and the microcontroller (28) utilizes the biometric feedback for controlling the control valve (36) using smart control algorithms running on the microcontroller (28) thus controlling the rate of drug injection. The illustrated sensor (46) is a pair of sensors for determining respiratory impairment (i.e. low levels of oxygenated hemoglobin in the blood of the wearer) as assessed by a reflectance oximeter, or oxygen-sensor The illustrated embodiment includes two of the sensors (46) located on opposite sides of the injection cylinder (16). The reflectance pulse oximeter (46) serves as a sensor which continuously feeds physiological data to the microcontroller (28) in the form of percent saturation of hemoglobin with oxygen (SpO2). The microcontroller (28) is preferably programmed to be activated at a SpO2 of less than, or equal to 90%. The control system preferably further includes a transmitter for sending a “911” signal to first responders of a possible overdose and a GPS tracking mechanism send first responders signals indicating the wearer's location. The microcontroller (28) is provided with a suitable processor, memory, and algorithms for controlling operation of the wearable device (10) as described herein. It is noted that the control system can alternatively have any other suitable configuration.

In operation, the wearable device (10) is secured to the skin of the wearer at a desired location using the adhesive layer (14). The sensors (46) monitors the wearer's physiological parameters or oxygen level and when the microcontroller (28) determines there is an event necessitating administration of the medication, the microcontroller (28) activates a trigger. Upon trigger by the microcontroller (28), the control valve (36) is opened by the microcontroller (28) and the compressed air enters the upper end of the injection cylinder (16) pushing the plunger (24) downward and thus pushing the injection piston (18) downward. Thus the hypodermic needle(s) (26) move downward until the hypodermic needle(s) (26) is fully inserted into the tissue of the wearer. At this stage, the plunger (24) moves downward in the fluid compartment 920) and the drug/fluid flows into the tissue of the wearer through the hypodermic needle(s) 926). This sequence occurs because there is less resistance to downward movement of the injection piston (18) than the resistance to downward movement of the plunger 924) into the fluid compartment (20).

FIGS. 4 and 5 illustrate a wearable device for automatically injecting a fluid into a wearer of the wearable device according to a second a second embodiment of the present invention. The wearable device includes an enclosure holding a plurality of vials each holding a different fluid to be injected into the wearer, an attachment for securing the enclosure to the wearer, a plurality of vial plungers each within a separate one of the plurality of vials and configured to selectively move the fluid out of the opposite end of the separate one of the plurality of vials when a force is applied to the vial plunger, a plurality of cartridges of compressed gas, and a plurality of electric valves each associated with a separate one of the plurality of cartridges and configured for selectively allowing flow of the compressed gas from the associated cartridge to the associated plunger to supply a force on the associated plunger, a plurality of needle cylinders each configured to receive the fluid from a separate one of the vials. Each of the plurality of needle cylinders includes a plunger movable within the associated needle cylinder and carrying at least one hypodermic needle for insertion into the wearer. The wearable device further includes a plurality of fluid conduits each connecting an outlet of a separate one of the vials to the associated at least one hypodermic needle, and a plurality of compressed-gas conduits each connecting one of the plurality of electric valves with an separate one of the plurality of needle cylinders to selectively move the associated needle piston to insert the at least one hypodermic needle into the wearer when compressed gas is supplied to the associate one of the plurality of vials.

The illustrated wearable includes three vials of fluid for injection but can alternatively have two vials of fluid or more than three vials of different fluid. For example, an embodiment with two vials could inject both a pain killer such as fentanyl and an antidote for overdose such as NARCAN. For example, an embodiment with three or more vials could inject three or more antidotes for biological or chemical weapons.

The wearable device includes the control system as described above. Each vial has a separate control valve for selectively providing compressed gas to the inlet of the vial to drive a vial plunger within the vial. Each of vial plungers include a metering system connected to the configured for monitoring the amount of the fluid injected through the at least one hypodermic needle. The compressed-gas conduits are each tubes and extend from the outlets of the control valves to an upper portion of the needle cylinders above the plungers to selectively provide compressed gas to downwardly drive the needle plunger and the hypodermic needle(s) through the protective membrane in into the wearer. The fluid conduits are each flexible tubes and extend from the outlets of the vials to the top of the plungers and the hypodermic needles to provide the fluid from the vials to the hypodermic needles. It is noted that the wearable devices can alternatively have any other suitable configuration.

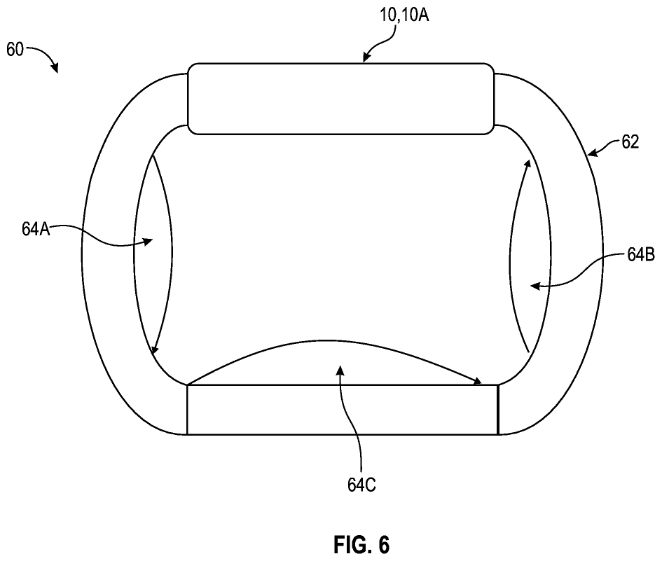

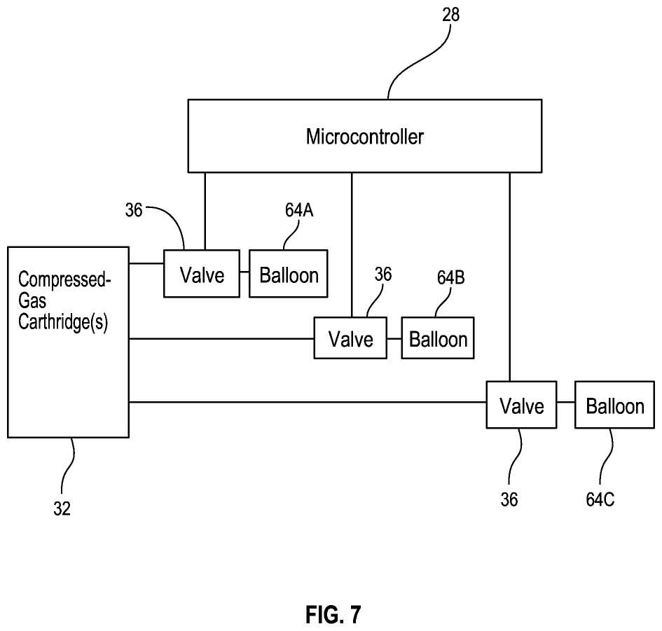

FIGS. 6 and 7 illustrate a system for automatically injecting a fluid into a wearer of the system. The system comprises a wearable dispenser for automatically injecting a fluid into the wearer and including a container for holding the fluid and at least one hypodermic needle for insertion into the wearer, a band for securing the wearable dispenser to the wearer and including at least one inflatable balloon, at least one cartridge of compressed gas, and at least one electric valve configured for selectively allowing flow of the compressed gas from the at least one cartridge to the at least one inflatable balloon to inflate the at least one inflatable balloon. The wearable dispenser can be either of the above-described wearable devices or any other suitable wearable dispenser. The band is preferably adapted to be worn about the wearers wrist or lower arm but can alternatively be configures to be worn about an upper arm portion, a lower leg, an upper leg portion or any other suitable location.

The illustrated band includes three of the inflatable balloons but any other suitable quantity can be utilized. A first inflatable balloon is located on a lateral side of the band and a second inflatable balloon is located on the opposite lateral side of the band. The first and second inflatable balloons can be used to reposition the dispensing device to avoid the hypodermic needle from hitting a bone. A third inflatable balloon is located opposite the wearable device that can be used to tighten the wearable device against the wearer's skin. At least one compressed-gas cartridge is connected to the inflatable balloons via separate electric valves so that the inflatable balloons can be individually inflated. Alternative a single control valve can be used but each of the inflatable balloons are controlled together. The control valves are controlled by the microcontroller. Preferably, the wearable device r automatically causes the at least one cartridge to inflate the third inflatable balloon upon activation of the wearable device. It is noted that the inflatable balloons can alternatively have any other suitable configuration.

Any of the features or attributes of the above-described embodiments and variations can be used in combination with any of the other features and attributes of the above-described embodiments and variations as desired.

From the foregoing disclosure it will be apparent that the illustrated smart wearable devices for automatically injecting medicines deliver medicines on-demand and without human intervention. It is also apparent that these wearable devices store drugs until needed, and upon activation insert hypodermic needles to sterilely deliver a pre-set volume of drug into the wearer's subcutaneous tissue. Additionally it is apparent that the wearable devices can deliver one or more time dependent stages once activated with a single intervention, can be connected to a physiological monitoring system for automatic activation without human intervention, can be activated remotely by health professionals or others monitoring physiological metrics, and that infections are eliminated because the needles are only deployed when triggered. Furthermore, it is apparent that the wearable devices are extremely compact, unobtrusive and convenient to wear for long periods of time. Moreover, the smart wearable devices can be used in many different applications including, but not limited to, defense applications in which soldiers utilize smart the smart wearable devices to inject pain killers, and/or antidotes to chemical or biological agents.

From the foregoing disclosure and detailed description of certain preferred embodiments, it will be apparent that various modifications, additions and other alternative embodiments are possible without departing from the true scope and spirit of the present invention. The embodiments discussed were chosen and described to provide the best illustration of the principles of the present invention and its practical application to thereby enable one of ordinary skill in the art to utilize the invention in various embodiments and with various modifications as are suited to the particular use contemplated. All such modifications and variations are within the scope of the present invention as determined by the appended claims when interpreted in accordance with the benefit to which they are fairly, legally, and equitably entitled.

What is claimed is:

1. A wearable device for automatically injecting a fluid into a wearer of the wearable device, the wearable device comprising, in combination:
an enclosure;
an attachment for securing the enclosure to the wearer;
at least one injection cylinder within the enclosure;
an injection piston within the injection cylinder configured for linear movement within the injection cylinder;
wherein the injection piston includes open and closed ends and forms an internal fluid compartment containing the fluid to be injected;
a plunger within the fluid compartment between the open end of the injection piston and the fluid to be injected and selectively movable within the fluid compartment toward the closed end of the injection piston;
at least one hypodermic needle carried by the injection piston for insertion into the wearer and in fluid flow communication with the fluid to be injected within the internal fluid compartment; and
wherein a force on the plunger initially moves the injection piston to insert the at least one hypodermic needle into the wearer and then moves the plunger within the fluid compartment to inject at least a portion of the fluid through the at least one hypodermic needle.

2. The wearable device according to claim 1, wherein the attachment includes a layer of adhesive on an outer surface of the enclosure.

3. The wearable device according to claim 1, wherein the attachment includes a band having at least one selectively inflatable balloon.

4. The wearable device according to claim 1, wherein the at least one hypodermic needle is secured to the closed end of the injection piston.

5. The wearable device according to claim 1, wherein a lower end of the injection cylinder is closed by a material pierceable by the at least one hypodermic needle.

6. The wearable device according to claim 1, wherein there is a plurality of the at least one hypodermic needle.

7. The wearable device according to claim 1, further comprising a metering system configured for monitoring the amount of the fluid injected through the at least one hypodermic needle.

8. The wearable device according to claim 1, further comprising a cartridge of compressed gas, and an electric valve selectively allowing flow of the compressed gas from the cartridge to within the injection cylinder above the injection piston to supply a force on the plunger.

9. The wearable device according to claim 1, further comprising a microcontroller configured to control the electric valve and a battery for powering the microcontroller and the electric valve.

10. A wearable device for automatically injecting a fluid into a wearer of the wearable device, the wearable device comprising, in combination:
an enclosure holding a plurality of vials each holding a different fluid to be injected into the wearer;
an attachment for securing the enclosure to the wearer;
a plurality of vial plungers each within a separate one of the plurality of vials and configured to selectively move the fluid out of the opposite end of the separate one of the plurality of vials when a force is applied to the vial plunger;
a plurality of cartridges of compressed gas, and a plurality of electric valves each associated with a separate one of the plurality of cartridges and configured for selectively allowing flow of the compressed gas from the associated cartridge to the associated plunger to supply a force on the associated plunger;
a plurality of needle cylinders each configured to receive the fluid from a separate one of the vials;
wherein each of the plurality of needle cylinders includes a plunger movable within the associated needle cylinder and carrying at least one hypodermic needle for insertion into the wearer;
a plurality of fluid conduits each connecting an outlet of a separate one of the vials to the associated at least one hypodermic needle; and
a plurality of compressed-gas conduits each connecting one of the plurality of electric valves with an separate one of the plurality of needle cylinders to selectively move the associated needle piston to insert the at least one hypodermic needle into the wearer when compressed gas is supplied to the associate one of the plurality of vials.

11. The wearable device according to claim 10, wherein the fluid conduits are each flexible tubes.

12. The wearable device according to claim 10, wherein the compressed-gas conduits are each tubes.

13. The wearable device according to claim 10, wherein there are at least two vials.

14. The wearable device according to claim 10, wherein the attachment includes a band.

15. The wearable device according to claim 10, further comprising a metering system configured for monitoring the amount of the fluid injected through the at least one hypodermic needle.

16. A system for automatically injecting a fluid into a wearer of the system, the system comprising, in combination:
a wearable dispenser for automatically injecting a fluid into the wearer and including a container for holding the fluid and at least one hypodermic needle for insertion into the wearer;
a band for securing the wearable dispenser to the wearer and including at least one inflatable balloon.
at least one cartridge of compressed gas, and
at least one electric valve configured for selectively allowing flow of the compressed gas from the at least one cartridge to the at least one inflatable balloon to inflate the inflatable balloon.

17. The system according to claim 16, wherein activation the wearable dispenser automatically causes the at least one cartridge to inflate the at least one inflatable balloon.

18. The system according to claim 16, wherein the at least one inflatable balloon is located opposite the wearable device.

19. The system according to claim 16, wherein the at least one inflatable balloon includes a first inflatable balloon one lateral side of the band and a second inflatable balloon located on the opposite lateral side of the band.

20. The system according to claim 19, wherein the at least one inflatable balloon further includes a third inflatable balloon located opposite the wearable device.
